(19) United States
(12) Patent Application Publication
(10) Pub. No.: US 2014/0205589 A1
(43) Pub. Date: Jul. 24, 2014

(54) NEW STABLE ANESTHETIC COMPOSITION FOR REDUCING SKIN REACTIONS

(71) Applicant: Thibaud Portal, Opio (FR)

(72) Inventor: Thibaud Portal, Opio (FR)

(73) Assignee: GALDERMA RESEARCH & DEVELOPMENT, Biot (FR)

(21) Appl. No.: 14/130,239

(22) PCT Filed: Jun. 29, 2012
(86) PCT No.: PCT/EP2012/062731

§ 371 Date: Mar. 31, 2014

Related U.S. Application Data
(60) Provisional application No. 61/502,480, filed on Jun. 29, 2011.

(30) Foreign Application Priority Data
Jul. 5, 2011 (FR) 1156041

(51) Int. Cl. A61K 31/498; A61K 9/107; A61K 45/06; A61K 31/167; A61K 31/245

(52) U.S. Cl. CPC A61K 31/498; A61K 31/167; A61K 31/245; A61K 45/06; A61K 9/107
USPC 424/94.67; 514/249

(57) ABSTRACT

A composition is described with reduced degradation rate and/or improved stability of its components. The composition can alleviate or even annihilate cutaneous reactions and can include an emulsion with an oil phase and an aqueous phase, wherein the oil phase can be a eutectic mixture of at least one anesthetic compound and at least one adrenergic receptor agonist. Methods of using such a composition are also described.

The present invention is in the dermatological domain.

The present invention provides a composition with reduced degradation rate and/or improved stability of its components and capable of decreasing or alleviating or even annihilate cutaneous reactions comprising an emulsion with an oil phase and an aqueous phase, said oil phase being a eutectic mixture of at least one anesthetic compound and at least one adrenergic receptor agonist. The invention also addresses to said composition further comprising polyvinyl alcohol and to said compositions further comprising at least one active agent.

It is well known that any aggression of the skin leads to a reaction of the latter at least uncomfortable, but often painful for the person who suffers. This is particularly true in the field of aesthetic surgery.Superficial bruising and, to a lesser extent, bleeding are not uncommon consequences (reported on average, about one-third of the time) of many aesthetic procedures, including injections of dermal fillers, Botulinum toxins and laser resurfacing.

More significant bruising occurs with surgical procedures such as liposuction, breast augmentations/lifts, face lifts and tummy tucks.

The management of secondary immediate reactions due to subcutaneous or intradermic injection, particularly of fillers, with vascular damages or vascular breaking wall inducing ecchymosis, bruising, leakage of blood components having immediate action on inflammation setting up, redness and oedema, are of particular interest.

Although bruising and bleeding, as well as redness and erythema are not generally considered a big problem, most physicians prepare their patients for this possibility by alerting them to it prior to the procedure. Particularly, physicians often caution against using aspirin or other anticoagulant drugs before and after the procedure, extensively use ice packs immediately after the procedure and quite commonly recommend Arnica, an herb used to promote healing. This kind of drawbacks may discourage some patients and particularly towards aesthetic procedures. In particular with regards to the consequences of bruising/bleeding, Physicians report that one of the most significant concerns for patients is the amount of “downtime” and when bruising occurs, patients prefer to stay home rather than return to work and social activities.

It is also well known that aesthetic procedures such as injection or laser surfacing or surgery, particularly skin surgery, or debridement (debridation from time to time of chronic ulcer surfaces, surfaces that have scabs and dead tissues (i. e. recovering burn wound)) can induce cutaneous reactions like bruising, bleeding, ecchymosis, erythema, oedema, necrosis, ulceration, swelling and/or inflammation and/or intense pain.

It is desirable to non invasively anesthetize the skin before some painful medical procedures, such as injections, cannulations, skin grafts, biopsies, minor superficial surgeries, and the like.

General analgesia, intravenous narcotic analgesics, regional nerve block by injection, and epidural anesthesia may be used to control the pain associated with aesthetic procedures such as injection or laser surfacing or surgery, particularly skin surgery, or debridement, cutaneous reactions like bruising, bleeding, ecchymosis, erythema, oedema, necrosis, ulceration, swelling and/or inflammation and/or intense pain such as those induced by some painful medical procedures, such as injections, cannulations, skin grafts, biopsies, minor superficial surgeries, and the like.

However, delivery of a general analgesic, regional nerve block by injection, epidural, or intravenous analgesic typically requires specially trained medical personnel and/or special medical equipment to administer.

The procedures also expose patients to significant risks and expose care givers to significant liability.

Applying analgesic formulation in which most of the active drug is dissolved onto skin lacking stratum corneum may result in dangerously rapid absorption of the drug and short duration of the effect. Some local anesthetic agents used in the prior art formulations to noninvasively anesthetize or provide analgesia to human body surfaces and tissues under the surface have significant limitations. Some commonly used local anesthetics, such as lidocaine have relatively limited penetration and sustain the analgesic effect for a relatively short period of time.

It would be particularly interesting to develop formulations and methods for non-invasive and convenient way to anesthetize the skin to prevent discomfort and/or pain in any future intervention on the skin, and at the same time allowing to prepare the skin to prevent or treat any adverse skin reactions resulting from such intervention as those described previously and particularly as bruising, bleeding, erythema or edema. It would also be advantageous to develop such compositions that could quickly deliver transdermally and simultaneously an anesthetic and an agent capable of alleviating or decreasing or even annihilating all consecutive reactions to such intervention, said composition having the characteristic of being easily removed.

Thus, it would be advantageous to develop methods in which the formulation is in the less-than solid form, such as a paste, gel, ointment, cream or solution, before being applied onto a human body surface and then the formulation can be converted into a coherent, solidified gel by a certain mechanism during the application to facilitate removal.

Here and after “less-than-solid phase,” unless specifically described otherwise, describes a form that is not as hard and as coherent as a solidified gel. Examples of such “less-than-solid” substances include toothpaste, cream, ointment, etc. One common property of these “less-than-solid” substances is that the substance is not strongly cohesive, or in other words, the substance is a liquid or a highly viscous fluid. In practical terms, a “less-than-solid” substance is a substance that one cannot grab and lift as a cohesive whole.

The present invention intended to provide such composition.

The applicant has now demonstrated that a composition comprising an emulsion with an oil phase and an aqueous phase, particularly when said oil phase is a eutectic mixture of at least one anesthetic compound and at least one adrenergic receptor agonist can be stable and can permit reduced degradation rate and/or improved stability of its components and can be capable of decreasing or alleviating or even annihilate cutaneous reactions.

Thus the invention relates to a composition with reduced degradation rate and/or improved stability of its components and for decreasing of alleviating or even annihilating cutaneous reactions comprising an emulsion with an oil phase and an aqueous phase, said oil phase comprising at least one anesthetic compound and at least one adrenergic receptor agonist.

In a preferred embodiment according to the invention, said oil phase can be a eutectic mixture of at least one anesthetic compound and at least one adrenergic receptor agonist. According to the invention a eutectic composition is a single mixture of chemical compounds or elements that solidifies at a lower temperature than any other composition. The temperature is known as the eutectic temperature.

In another particular embodiment the invention also relates to a composition with reduced degradation rate and/or improved stability of its components and for decreasing of alleviating or even annihilating cutaneous reactions comprising:
a. an emulsion with an oil phase and an aqueous phase, comprising at least one anesthetic compound and at least one adrenergic receptor agonist, and
b. polyvinyl alcohol.

In a particular embodiment of this embodiment according to the invention, said oil phase can be a eutectic mixture of at least one anesthetic compound and at least one adrenergic receptor agonist.

Polyvinyl alcohol is the polymer that can convert a cream into a solid after enough of the water in the formulation is evaporated.

According to the invention said polyvinyl alcohol can have an initial concentration in the composition such that the composition is in a less than solid state, wherein in use the polyvinyl alcohol converts the composition into a coherent, peelable solid state.

According to the invention the polyvinyl alcohol can be present in the composition from about 1% to about 5%, preferably from about 2% to about 4% in weight.

Regardless of the embodiment of the invention said anesthetic compound can be at least one local anesthetic or itself a eutectic mixture of at least two local anesthetics, advantageously selected from the group of lidocaine, tetracaine, prilocaine, benzocaine, bupivacaine, mepivacaine, dibucaine, etidocaine, butacaine, cyclomethycaine, hexylcaine, proparacaine, and lopivacaine, preferentially a mixture of lidocaine and tetracaine, more preferably a eutectic of lidocaine and tetracaine.

According to the invention, said anesthetic can represent at least 5% by weight of the composition, advantageously 10% preferably 14% by weight of the composition.

The second component of the claimed composition is an adrenergic receptor agonist.

Adrenergic receptor agonists are known to bind and activate the adrenergic receptors.

The adrenergic receptors (or adrenoceptors) are a class of metabotropic G protein-coupled receptors that are targets of the catecholamines, especially noradrenaline (norepinephrine) and adrenaline (epinephrine). Although dopamine is a catecholamine, its receptors are in a different category. There are two main groups of adrenergic receptors, α and β, with several subtypes.
α a receptors have the subtypes α₁ (a G_{q} coupled receptor) and α₂ (a G₁ coupled receptor). Phenylephrine is a selective agonist of the α receptor.
β receptors have the subtypes β₁, β₂ and β₃. All three are linked to Gₛ proteins (although β₂ also couples to Gi),^{[1]} which in turn are linked to adenylate cyclase. Agonist binding thus causes a rise in the intracellular concentration of the second messenger cAMP. Downstream effectors of cAMP include cAMP-dependent protein kinase (PKA), which mediates some of the intracellular events following hormone binding. Isoprenaline is a selective agonist.

As it is well known in the art, adrenergic receptors encompass both α and β receptors. Among α adrenoreceptors, α1 and α2 receptors were distinguished in the 1970's. During the same decade, α2 receptors were found to occur on vascular smooth muscles and exhibit mediation of vasoconstrictor response (“Subtypes of functional α₁- and α₂-adrenoceptors” J R Docherty; European Journal of Pharmacology 361 (1998) 1-15). Thus, molecules exhibiting a adrenergic agonism, advantageously α2 adrenergic agonism, possess peripheral vasoconstrictive activity.

Agonists to be used in the claimed composition can be directed to α and/or β receptors. However, because of their possible side-effects, molecules exhibiting β adrenergic agonism, are advantageously disclaimed. In the rest of the application, a molecule having no affinity for the β adrenergic receptors will be named “an α-adrenergic receptor agonist”.

Among the a receptors, the agonist can be an agonist of both α1 and α2 receptors, or can be specific for α1 or α2. Preferably, the chosen molecule displays more affinity for the α2 than for the al receptor, and will generally be named, in the rest of the application, “an α2 adrenergic receptor agonist”.

In a preferred embodiment, the adrenergic receptor agonist is an adrenergic receptor agonist α2, preferably brimonidine.

Agonists of the α2 adrenoceptors have been used therapeutically for a number of conditions including hypertension, congestive heart failure, angina pectoris, spasticity, glaucoma, diarrhea, and for the suppression of opiate withdrawal symptoms (J. P. Heible and R. R. Ruffolo Therapeutic Applications of Agents Interacting with a-Adrenoceptors, p. 180-206 in Progress in Basic and Clinical Pharmacology Vol. 8, P. Lomax and E. S. Vesell Ed., Karger, 1991).

Adrenoceptor agonists such as clonidine have been primarily used orally, though a patch formulation is known. The α2 agonists are known to mediate vasoconstriction both in the core and periphery of a patient. In particular α2 adrenoceptor agonists are known to cause vasoconstriction of peripheral arterioles, in response to stimulation due to cold or stress.

A number of patents describe the use of brimonidine for treating ophthalmic conditions and eye diseases. In Canadian patent No. CA2326690, there is described the use of topical ophthalmic preparations for use only in the eyes, to treat eye diseases.

As already said above, the most preferred compound in the context of the invention is (5-Bromo-quinoxalin-6-yl)-(4, 5-dihydro-IH-imidazol-2-yl)-amine (commonly referred to as brimonidine) and pharmaceutically acceptable salts thereof, particularly the tartrate salt.

Other compounds known to be α2 adrenoceptor agonists and which can be used in the frame of the present invention are: clonidine, apoclonidine.

More generally, other compounds which are α adrenoceptor agonists are: synephrine, octodrine, vasopressine and analogs, ornipressine, midodrine, phenylephrine, xylometazoline, oxymetazoline, norepinephrine, methoxamine.

Compounds which have also an affinity for the β receptors but which can be used in certain conditions are: epinephrine, ephedrine, etilefrine.

Of course, the pharmaceutically acceptable salts of all these compounds are also encompassed.

According to the instant invention, the term “pharmaceutically acceptable salt (s)”, as used herein, means those salts of compounds of the invention that are safe and effective for topical use in mammals and that possess the desired biological activity. Pharmaceutically acceptable salts include salts of acidic or basic groups present in the compounds of the invention.

Pharmaceutically acceptable acid salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i. e., 1, 1′-methylene-bis-(2-hydroxy-3-naphthoate)) salts. Certain compounds of the invention can form pharmaceutically acceptable salts with various amino acids.

Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. Sci. 1-19 (1977).

According to the invention, said adrenergic receptor agonist, preferably brimonidine, represents between 0.01% and 5%, by weight of the composition, preferentially between 0.02 et 1%, and more preferably between 0.05 et 0.5% by weight of the composition.

According to the invention, said emulsion can be thickened such that it is substantially non-flowable and cohesive at ambient temperature.

In an another embodiment of the invention said composition can further include at least one compound that is a pH regulating agent(s), a colouring agent(s), a permeation enhancing agent(s), an emulsifying agent, a gelling agent, a thickening agent or a combination thereof. Preferably said in composition said emulsion is gelled. This means that said gelled emulsion rapidly melts or significantly softens when heated to greater than about 30° C., and that said gelled emulsion does not melt or significantly soften when heated to about 30° C.

The control of water loss and retention is an important part of the present invention. It is believed that only the drug molecules that are dissolved in the water of the formulation can effectively penetrate the skin.

According to the invention water has to be retained long enough to allow sufficient amounts of the drug to be delivered into the skin within a reasonable time, while at the same time permitting enough water to be lost by evaporation so that the formulation becomes a soft solid that can be easily peeled off the skin after the numbing effect is achieved.

Water retaining ability can be provided by Water Lock™ and glycerol. Water Lock™ also contributes to the viscosity of the formulation on the skin.

Glycerol serves as a plasticizing agent, which allows the formulation to become a soft, flexible solid, rather than a rigid one, after the evaporation of the water. Glycerol also has a tendency to retain water.

Water Lock is used to retain water as well as to increase the viscosity, so that the formulation has a minimal ability to flow.

Thickness of the formulation on the skin can be an important factor of the present invention. If the layer of formulation on the skin is too thin, the formulation will dry out before sufficient amounts of the drug are delivered. If the layer is too thick, the portion in contact with the skin will remain as a cream, while the outside layer exposed to air may solidify. The thickness of the layer should be adjusted to correspond with the appropriate water loss and water retention requirements of a given formulation and given therapeutic need. For example a thicker layer of the formulation should be used to achieve anesthesia with greater depth. That is because the formulation in contact with the skin can retain water for a longer period of time, and hence deliver the drug for longer time, if the layer is thicker.

For example for a composition containing local anesthetic, the optimal thickness should be somewhere between 0.5-3 mm, more likely between 1-2 mm, depending on the length of time it takes to anesthetize the particular skin, and how dry the ambient air is.

One of the advantages of the present invention is that it obviates the need to remove the cream from the skin by extensive washing or cleansing of the skin.

Washing and cleansing the skin takes extra effort and time. It can also irritate the skin and compromised body surfaces of the skin. Controlling water retention according to the present invention obviates the need for time consuming clean-up of the drug formulation, while permitting adequate delivery of the drug.

To deliver a drug the drug formulation can be applied to the skin at a desired delivery location. The drug formulation can be applied in a layer having a substantially consistent thickness. For drug formulation that use water as a vehicle for skin permeation, the drug can continue to be delivered as water evaporates until most of the water the is evaporated and the formulation is a soft peelable solid. When the desired anesthetic effect is achieved, the solid gel is peeled off the skin area, leaving virtually no residual mess on the skin. The skin area is anesthetized and if desired can be subjected to a medical treatment or procedure. For drugs that can penetrate the skin without having to dissolve in water first, drug delivery can continue after the water is evaporated.

In another particular embodiment the invention also relates to a composition with reduced degradation rate and/or improved stability of its components and for decreasing of alleviating or even annihilating cutaneous reactions as previously described, (an emulsion with an oil phase and an aqueous phase, eventually said oil phase being a eutectic mixture of at least one anesthetic compound and at least one adrenergic receptor agonist, with or without polyvinyl alcohol) further comprising at least one active agent.

According to the invention, said active agent can be chosen from Antivirals (e. g. acyclovir); Antibiotics (e. g. bacitracin, chloramphenicol, clindamycin, erythromycin, gentamicin, mupirocin, neomycin, tetracylcines); Antifungals (e. g. amphotericin B, benzoic acid, salicylic acid, butaconazole, ciclopirox, clioquinol, clotrimazole, econazole nitrate, haloprogin. ketoconazole, micronazole, naftifine, nystatin, oxiconazole, sodium thiosulfate, terconazole, triacetin, undecyclenic acid, and undecylenate salts); Other Antiseptics (e. g. benzalkonium chloride, hexachlorophene. iodine, mafenide, metronidazole, nitrofurazone, selenium sulfide, slyer sulfadiazine); Anti-inflammatory Agents (e. g. corticosteroids); Antiprurities; Cell stimulants and proliferants (e. g. tretinoin for treating acne); Emollients (e. g. vitamins A, D); Agents for Treating Necrolic Tissues and Dermal Ulcers or Used in Debridement (e. g. collagenase, fibrinolysin, desoxribonuclease, sutilains); Anti-Skin Cancer,'Anti-Kefatosis Ager. is (e. g. fluoronracil; Wound Cleansing Agents (e. g. dextranomer); Agents for Promoting Hair Growth (e. g. minoxidil); Depigmenting Agents (e. g. hydroquinc'ne, monobenzcne); Sunscreen Agents and Coemical Sunscreen Agents (e. g. aminobenzoic acid derivatives such as aminobenzoic acid and menthyl ambranilate; benzophenone derivatives such as dioxybenzone and oxybenzone; salicylate derivatives; cinnamic acid derivatives; gigalloyi moleate) and Opaque Physical Sunscreen Agents (e. g. red petrolatum, titanium dioxide, zinc oxide); Other Dermatological and Pharmaceutical Agents such as psoriasis drugs (e. g. anthralin, calcipotriens), drugs for promoting wound healing, drugs for treating warts and moles, drugs for treating ulcerated skin surfaces, drugs used on newborn babies that need to be delivered in a patch form (the adhesive in patches may be too agressive for newborn babies'skin); drugs that are applied to mucosa (e. g. alprostadil and other drugs for treating male erectile dysfunction (on penis tip and/or into urethra)), and drugs for treating mucosal warts (e. g. imiquimod).

The invention also relates to a method for decreasing of alleviating or even annihilating cutaneous reactions wherein one applies to an individual in need, a composition comprising an emulsion with an oil phase and an aqueous phase, said oil phase comprising at least one anesthetic compound and at least one adrenergic receptor agonist as previously described herein before.

In a preferred embodiment according to the invention, said oil phase can be a eutectic mixture of at least one anesthetic compound and at least one adrenergic receptor agonist.

According to the invention said cutaneous reactions can be for example selected from the following: bruising, bleeding, ecchymosis, erythema, oedema, redness, necrosis, ulceration, swelling and/or inflammation and/or intense pain, vascular damages or vascular breaking wall inducing ecchymosis, leakage of blood components having immediate action on inflammation setting up.

One of the main aims of the invention, but not the only one, is a method for decreasing of alleviating or even annihilating cutaneous reactions, preferably before injection of at least one filler, or toxin, such as for example Botulinum toxin.

Filler is generally defined and must be understood according to the invention as a biomaterial able to fill dermal tissues. In this context, same compounds like polyacrylamid gels, polymethylmethacrylate (PMMA) particles or silicones can be used. The most preferred compounds are resorbable molecules such as hyaluronic acid, collagen, alginate, dextran, elastine or polyurethane gels. Therefore and advantageously, the filler is hyaluronic acid or a pharmaceutically acceptable salt or derivative thereof, particularly the sodium or potassium salt. Hyaluronic acid can be used under different forms: salts thereof, derivatives thereof such as esters or amides, in a linear form or cross-linked. In particular, the molecular weight, typically comprised between 500 kDa and 5 000 kDa, and the degree of cross-linking depends on the application, especially on the depth of the wrinkles to be filled.

The invention also relates to a method for decreasing of alleviating or even annihilating, bruising and, to a lesser extent, bleeding and particularly in aesthetic procedures including injection and laser resurfacing, by providing to an individual in need thereof, a composition with reduced degradation rate and/or improved stability of its components and for decreasing of alleviating or even annihilating cutaneous reactions, as previously described herein before.

Regardless of the method of the invention the anesthetic compound and the adrenergic receptor agonist are formulated for simultaneous application in a single composition according to the invention.

Transdermal drug delivery rates and doses can be determined primarily by the dimensional surface area of the body surface that can be in contact with the drug formulation. Drug delivery systems which do not provide the ability to control the surface area covered by the formulation make it difficult to control the dose or rate of drug delivery. Drug delivery systems which do not allow the surface area to be varied in a regulated manner make it difficult to vary dose and rate according to varying circumstances.

The present invention provides the ability to vary and to control the surface area in contact with the formulation. By providing a formulation which converts to a solid after application as a less-than-solid formulation, the present invention allows the surface area to be varied to suit different applications, but, upon the formulation's conversion, allows the formulation to maintain the desired surface. Once solidified, the drug does not flow away from the administration to be absorbed elsewhere and thereby change the overall dose and rate of delivery. Allowing the user to chose from a variety of patches having different surface areas and fill the patches with a drug formulation that will convert to a solid provides similar benefits.

In addition to the above, the following examples are provided to illustrate particular embodiments and not to limit the scope of the invention.

### EXAMPLE 1

| | | Weight | |
|---|---|---|---|
| | Ref. to | percentage | |
| Component | standard | (% w/w) | Function |
| Lidocaine base | Ph. Eur. | 7.00 | Active anesthetic agent |
| Brimonidine | | 0.3 | Active |
| Dibasic calcium | Ph Eur | 36.00 | Thickening agent |
| phosphate, anhydrous | | | |
| Purified water | Ph Eur | Qsp 100 | Solvent |
| Polyvinyl alcohol (Low | USP | 12.00 | Polymer |
| molecular weight) | | | |
| White petrolatum | Ph Eur | 10.00 | Emollient |
| Sorbitan monopalmitate | NF | 2.00 | Emulsifying agent |
| (Span ® 40) | | | |
| Methylparaben | Ph Eur | 0.05 | Preservative |
| Propylparaben | Ph Eur | 0.01 | Preservative |

### EXAMPLE 2

| | | Weight | |
|---|---|---|---|
| | Ref. to | percentage | |
| Component | standard | (% w/w) | Function |
| Tetracaine base | USP | 7.00 | Active anesthetic agent |
| Brimonidine | | 0.2 | Active |
| Dibasic calcium | Ph Eur | 36.00 | Thickening agent |
| phosphate, anhydrous | | | |
| Purified water | Ph Eur | Qsp 100 | Solvent |
| Polyvinyl alcohol (Low | USP | 12.00 | Polymer |
| molecular weight) | | | |
| White petrolatum | Ph Eur | 10.00 | Emollient |
| Sorbitan monopalmitate | NF | 2.00 | Emulsifying agent |
| (Span ® 40) | | | |
| Methylparaben | Ph Eur | 0.05 | Preservative |
| Propylparaben | Ph Eur | 0.01 | Preservative |

### EXAMPLE 3

| | | Weight | |
|---|---|---|---|
| | Ref. to | percentage | |
| Component | standard | (% w/w) | Function |
| Lidocaine base | Ph. Eur. | 7.00 | Active anesthetic agent |
| Tetracaine base | USP | 7.00 | Active anesthetic agent |
| Brimonidine | | 0.3 | Active |
| Dibasic calcium | Ph Eur | 36.00 | Thickening agent |
| phosphate, anhydrous | | | |
| Purified water | Ph Eur | Qsp 100 | Solvent |
| Polyvinyl alcohol (Low | USP | 12.00 | Polymer |
| molecular weight) | | | |
| White petrolatum | Ph Eur | 10.00 | Emollient |
| Sorbitan monopalmitate | NF | 2.00 | Emulsifying agent |
| (Span ® 40) | | | |
| Methylparaben | Ph Eur | 0.05 | Preservative |
| Propylparaben | Ph Eur | 0.01 | Preservative |

1. A composition comprising an emulsion with an oil phase and an aqueous phase, said oil phase comprising at least one anesthetic compound and at least one adrenergic receptor agonist, wherein the composition reduces a degradation rate of and/or provides improved stability of its components and decreases, alleviates or even annihilates cutaneous reactions.

2. The composition of claim 1, wherein said oil phase is a eutectic mixture of at least one anesthetic compound and at least one adrenergic receptor agonist.

3. The composition of claim 1, wherein said anesthetic compound is at least one local anesthetic.

4. The composition of claim 1, wherein said anesthetic compound is itself a eutectic mixture of at least two local anesthetics.

5. The composition of claim 1, wherein said anesthetic is selected from the group consisting of lidocaine, tetracaine, prilocaine, benzocaine, bupivacaine, mepivacaine, dibucaine, etidocaine, butacaine, cyclomethycaine, hexylcaine, proparacaine, and lopivacaine.

6. The composition of claim 1, wherein said anesthetic is a mixture of lidocaine and tetracaine.

7. The composition of claim 1, wherein said anesthetic is a eutectic mixture of lidocaine and tetracaine.

8. The composition of claim 1, wherein said anesthetic represents at least 5% by weight of the composition.

9. The composition of claim 1, wherein said anesthetic represents at least 10% by weight of the composition.

10. The composition of claim 1, wherein said anesthetic represents at least 14% by weight of the composition.

11. The composition of claim 1, wherein said adrenergic receptor agonist is an adrenergic receptor agonist a-1 or a-2.

12. The composition of claim 1, wherein said adrenergic receptor agonist is selected from the group consisting of brimonidine clonidine, apoclonidine. synephrine, octodrine, vasopressine, omipressine, midodrine, phenylephrine, xylometazoline, oxymetazoline, norepinephrine, and methoxamine.

13. The composition of claim 1, wherein said adrenergic receptor agonist is brimonidine.

14. The composition of claim 1, wherein said adrenergic receptor agonist, represents between 0.01% and 5%, by weight of the composition.

15. The composition of claim 1, wherein said adrenergic receptor agonist, represents between 0.02% et and 1% by weight of the composition.

16. The composition of claim 1, wherein said adrenergic receptor agonist, represents between 0.05% and 0.5% by weight of the composition.

17. The composition of claim 1, wherein said emulsion is thickened such that it is substantially non-flowable and cohesive at ambient temperature.

18. The composition of claim 1, further comprising pH regulating agent(s), coloring agent(s), permeation enhancing agent(s), or a combination thereof.

19. The composition of claim 1, further comprising at least one compound that is an emulsifying agent, a gelling agent, or a thickening agent.

20. The composition of claim 1, wherein said emulsion is gelled.

21. The composition of claim 23, wherein said gelled emulsion rapidly melts or significantly softens when heated to greater than about 30° C.

22. The composition of claim 23, wherein said gelled emulsion does not melt or significantly soften when heated to about 30° C.

23. A composition comprising:
a. an emulsion with an oil phase and an aqueous phase, said oil phase being a eutectic mixture of at least one anesthetic compound and at least one adrenergic receptor agonist, and
b. polyvinyl alcohol, wherein the composition reduces a degradation rate of and/or provides improved stability of its components and decreases, alleviates or even annihilate cutaneous reactions.

24. The composition of claim 23, wherein said oil phase is a eutectic mixture of at least one anesthetic compound and at least one adrenergic receptor agonist.

25. The composition of claim 23, wherein said anesthetic compound is at least one local anesthetic.

26. The composition of claim 23, wherein said anesthetic compound is itself a eutectic mixture of at least two local anesthetics.

27. The composition of claim 23, wherein said anesthetic is selected from the group consisting of lidocaine, tetracaine, prilocaine, benzocaine, bupivacaine, mepivacaine, dibucaine, etidocaine, butacaine, cyclomethycaine, hexylcaine, proparacaine, and lopivacaine.

28. The composition of claim 23, wherein said anesthetic is a mixture of lidocaine and tetracaine.

29. The composition of claim 23, wherein said anesthetic is a eutectic mixture of lidocaine and tetracaine.

30. The composition of claim 23, wherein said anesthetic represents at least 5% by weight of the composition.

31. The composition of claim 23, wherein said anesthetic represents at least 260% by weight of the composition.

32. The composition of claim 23, wherein said anesthetic represents at least 264% by weight of the composition.

33. The composition of claim 23, wherein said adrenergic receptor agonist is an adrenergic receptor agonist α-1 or α-2.

34. The composition of claim 23, wherein said adrenergic receptor agonist is selected from the group consisting of brimonidine clonidine, apoclonidine. synephrine, octodrine, vasopressine, ornipressine, midodrine, phenylephrine, xylometazoline, oxymetazoline, norepinephrine, and methoxamine.

35. The composition of claim 23, wherein said adrenergic receptor agonist is brimonidine.

36. The composition of claim 23, wherein said adrenergic receptor agonist represents between 0.01% and 5%, by weight of the composition.

37. The composition of claim 23, wherein said adrenergic receptor agonist represents between 0.02% and 1% by weight of the composition.

38. The composition of claim 23, wherein said adrenergic receptor agonist, represents between 0.05% and 0.5% by weight of the composition.

39. The composition of claim 23, further comprising at least one filler or at least one toxin.

40. The composition of claim 23, wherein said filler is selected from the group consisting of polyacrylamid gels, polymethylmethacrylate (PMMA) particles, silicones hyaluronic acid, collagen, alginate, dextran, elastine, and polyurethane gels.

41. The composition of claim 23, wherein said filler is hyaluronic acid or a pharmaceutically acceptable salt thereof.

42. The composition of claim 23, wherein said filler is a pharmaceutically acceptable hyaluronic acid sodium or potassium salt.

43. The composition of claim 23, wherein said emulsion is thickened such that it is substantially non-flowable and cohesive at ambient temperature.

44. The composition of claim 23, further including pH regulating agent(s), coloring agent(s), permeation enhancing agent(s), or a combination thereof.

45. The composition of claim 23, further including at least one compound that is an emulsifying agent, a gelling agent, or a thickening agent.

46. The composition of claim 23, wherein said emulsion is gelled.

47. The composition of claim 46, wherein said gelled emulsion rapidly melts or significantly softens when heated to greater than about 30° C.

48. The composition of claim 46, wherein said gelled emulsion does not melt or significantly soften when heated to about 30° C.

49. A method for decreasing, alleviating or even annihilating cutaneous reactions, the method comprising applying to an individual in need thereof, a composition comprising an emulsion with an oil phase and an aqueous phase, said oil phase comprising at least one anesthetic compound and at least one adrenergic receptor agonist as described in claim 1.

50. The method of claim 49, wherein said cutaneous reactions are due or will be due to an aesthetic procedure.

51. The method of claim 50, wherein said aesthetic procedure is injection or laser resurfacing.

52. The method of claim 49, wherein the cutaneous reactions are selected from the group consisting of: bruising, bleeding, ecchymosis, erythema, oedema, redness, necrosis, ulceration, swelling and/or inflammation and/or intense pain, and vascular damages or vascular breaking wall inducing ecchymosis, leakage of blood components having immediate action on inflammation setting up.

53. A method for decreasing, alleviating or even annihilating cutaneous reactions, the method comprising applying to an individual in need thereof the composition of claim 1, where the cutaneous reactions are due to injection of at least one filler, or toxin.

54. The method of claim 52, wherein said composition is applied before injection of at least one filler, or toxin.

55. The composition of claim 14, wherein said andrenergic receptor agonist is brimonidine.

56. The composition of claim 15, wherein said andrenergic receptor agonist is brimonidine.

57. The composition of claim 16, wherein said andrenergic receptor agonist is brimonidine.

58. The composition of claim 36, wherein said andrenergic receptor agonist is brimonidine.

59. The composition of claim 37, wherein said andrenergic receptor agonist is brimonidine.

60. The composition of claim 38, wherein said andrenergic receptor agonist is brimonidine

61. The composition of claim 39, wherein the at least one toxin is Botulinum toxin.

62. The method of claim 53, wherein the at least one toxin is Botulinum toxin.

63. The method of claim 54, wherein the at least one toxin is Botulinum toxin.
